(19) United States
(12) Patent Application Publication
(10) Pub. No.: US 2010/0112588 A1
(43) Pub. Date: May 6, 2010

(54) METHODS FOR SANGER SEQUENCING USING PARTICLE ASSOCIATED CLONAL AMPLICONS AND HIGHLY PARALLEL ELECTROPHORETIC SIZE-BASED SEPARATION

(75) Inventors: Javier Farinas, Los Altos, CA (US); Andrea Chow, Los Altos, CA (US); John Wallace Parce, Palo Alto, CA (US)

Correspondence Address: Javier Farinas, 1644 Kensington Ave., Los Altos, CA, 94024, US

(73) Assignee: CAERUS MOLECULAR DIAGNOSTICS, INC., Los Altos, CA (US)

(21) Appl. No.: 12/611,528

(22) Filed: Nov. 3, 2009

Related U.S. Application Data
(60) Provisional application No. 61/111,043, filed on Nov. 4, 2008.
(60) Provisional application No. 61/165,514, filed on Apr. 1, 2009.

(51) Int. Cl. C12Q 1/68

(52) U.S. Cl. 435/6

(57) ABSTRACT

Methods for highly parallel Sanger sequencing are discussed. In particular, provided herein are methods using particles to clonally amplify templates and to introduce the amplified nucleic acids into many parallel channels with a single template per channel. Once in the channels, the nucleic acids are separated by size using electrophoresis to produce long read length sequencing information. Methods involving optical detection of the size-separated nucleic acids and analysis of the resulting electropherograms to yield the sequences are disclosed.

### CROSS-REFERENCE TO RELATED APPLICATIONS

This application claims the benefit of PPA Ser. No. 61/111,043, filed Nov. 4, 2008 and PPA Ser. No. 61/165,514, filed Apr. 1, 2009 by the present inventors, which are incorporated by reference.

### FEDERALLY SPONSORED RESEARCH

Not applicable.

### SEQUENCE LISTING OR PROGRAM

Not applicable.

### BACKGROUND

1. Technical Field

The present disclosure is in the field of nucleic acid sequencing. In particular, described herein are methods for sequencing a very large number of clonally amplified nucleic acids in parallel with long read lengths.

2. Prior Art

Nucleic acid sequencing is an important part of medical research, diagnostics, industrial processing, crop and animal breeding, and many other fields. For example, sequencing is used to diagnose disease conditions, detect infectious organisms, identify individuals in forensic applications and discover disease-causing genes.

A commonly used method of nucleic acid sequencing is Sanger sequencing.¹ The Sanger method uses dideoxynucleotide triphosphates (ddNTPs) as DNA chain terminators to generate a set of nucleic acid fragments which differ in length by one nucleotide. The dideoxynucleotides (e.g. ddATP, ddGTP, ddCTP and ddTTP) which cause chain termination can be identified by labeling each dideoxynucleotide with a distinguishable detectable label.² The labeled DNA fragments are size separated by gel electrophoresis with single nucleotide resolution. Electrophoretic separation is performed in slab gels, capillaries or microfluidic devices using denaturing polyacrylamide-urea gels or other sieving polymer matrices. The DNA sequence is defined by the order in which the dideoxynucleotide terminated fragments appear. One of the drawbacks of Sanger sequencing is the large amount of sample preparation required to sequence nucleic acids which results in high cost.

Recently, new methods have been developed for ultra high throughput sequencing of nucleic acids based on highly parallel schemes which greatly reduce the per base cost of sequencing. Most of these new methods use an in vitro cloning step to generate many spatially localized copies of individual template nucleic acid molecules in a sample. For example, one method for generating a library of clonally amplified template molecules is emulsion PCR.³ A water-in-oil emulsion is formed such that the aqueous droplets dispersed in the oil phase contain amplification reagents such as polymerase chain reaction (PCR) reagents and limiting amounts of primer-coated beads and templates. The beads and templates are added in amounts such that most beads bind zero or one template. Additionally, most droplets have zero or one bead. A PCR reaction is carried out to amplify the templates. Usually, the amplicons are bound to the beads by primers covalently attached to the beads. After breaking the emulsion, the beads can be processed in parallel either in a sequencing-by-synthesis or ligation method to obtain sequence information.⁴ Another method for in vitro clonal amplification is “bridge PCR”, where fragments are amplified using primers attached to a solid surface.⁵ Both of these methods produce many physically isolated locations which each contain many copies of a single template.

Once clonal amplification is completed, various methods are used to read out a sequence. The 454 method uses a fiber optic slide with millions of individual wells for highly parallel pyrosequencing reactions.⁴ Another method involves incorporation of fluorescently labeled reversible terminator nucleotides into clonal amplicons distributed on the surface of a flow cell.⁶ Yet another method uses emulsion PCR to generate clonally amplified libraries which are deposited on a glass slide. A series of probe oligos are ligated to the bead-bound nucleic acids to read out the sequence.⁷

### ADVANTAGES

Despite the wide usage of Sanger sequencing and the host of newly developed high throughput sequencing methods, certain deficiencies persist in current nucleic acid sequencing methods. Some of the drawbacks of Sanger sequencing are the large amount of sample preparation required to sequence nucleic acids and the high per-base cost of sequencing. New high throughput sequencing methods can analyze millions or billions of clones in parallel but are often limited to obtaining 20-50 nucleotides of sequence information per clone. For some applications however, it is important to obtain hundreds or thousands of nucleotides of information per clone. For example, in de novo genome sequencing, long read lengths are required to close gaps.⁸ In another example, long read lengths are required to unambiguously detect linked mutations in HIV genotyping or HLA allelotyping applications.⁹ Next generation methods also typically have lower accuracy than Sanger Sequencing.

Thus, there remains a need for improved sequencing methods that are lower cost compared to conventional Sanger sequencing and that have longer read lengths and/or higher accuracy compared to current next generation sequencing methods.

### SUMMARY

The present teachings provide systems for measuring nucleic acid sequences using particle-based clonal amplification and injections, and high throughput electrophoretic size-based separation. This invention reduces the cost and time required to perform Sanger sequencing using conventional methods while maintaining the advantages of long read lengths and/or higher accuracy compared to next generation sequencing methods.

### DRAWINGS

FIG. 1. Schematic example of a bead showing dideoxy terminated fragments of varying size covalently attached to a bead by a photocleavable linker.

FIG. 2. Schematic example of a device with multiple channels connecting two reservoirs. Typical distances are: A 3-50 um, B 10-200 um and C 3-100 cm.

FIG. 3. Schematic showing alignment of particles with channel openings.

FIG. 4. Schematic showing part of a device with spatial traps. A particle suspension is flowed past the structures. The particles are trapped between the semicircular structure and the top of a flow cell.

FIG. 5. Schematic of part of a device showing spatial traps printed near the channel entrances. For example, binding of the beads to the traps can be mediated by biotin/avidin binding.

FIG. 6. A sequence of photographs illustrating the feasibility of the electrophoretic alignment process. A 25 um agarose bead was captured at the top of the capillary lumen using a field of 167 V/cm. The bead is highlighted by an arrow from panels (A) through (E). The time between each panel is 0.4 seconds.

FIG. 7. A sequence of fluorescence photomicrographs illustrates the process of DNA cleavage from beads using a UV source to break a photolytic linkage. The fragments are immediately injected into the associated channel for electrophoretic separation. (A): Bead-bound DNA captured at the beginning of a separation channel, (B): DNA cleaved from beads after UV exposure, (C): electric field switched on, (D) through (F): DNA fragments electrophoresing down the channel. The time between frames in D-F is 1 second.

### DETAILED DESCRIPTION

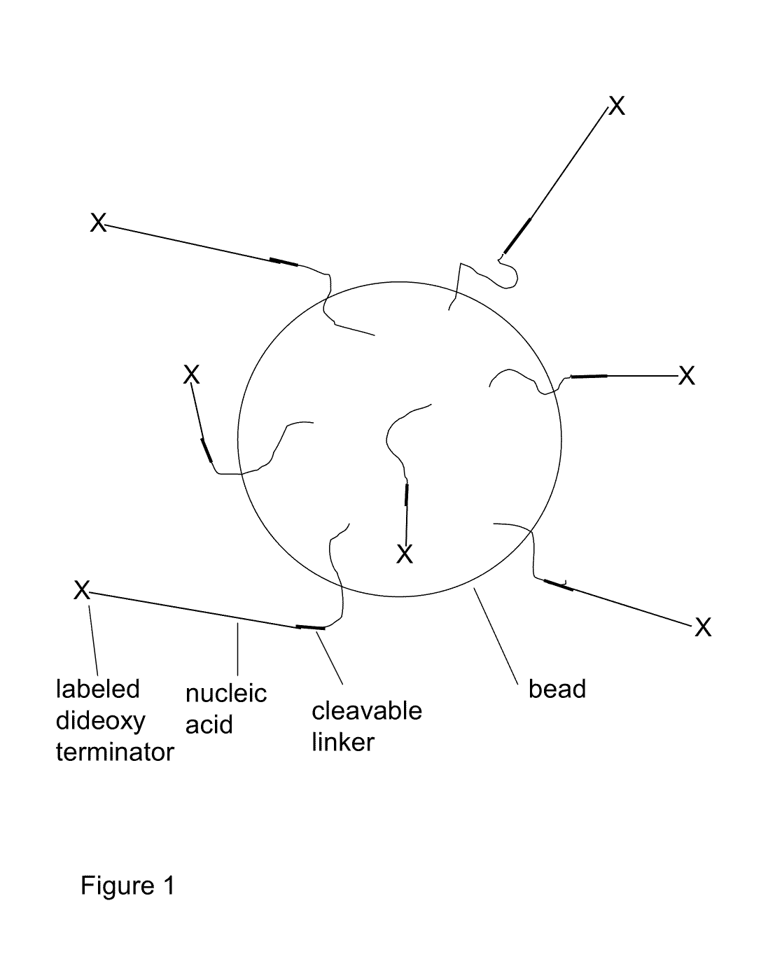

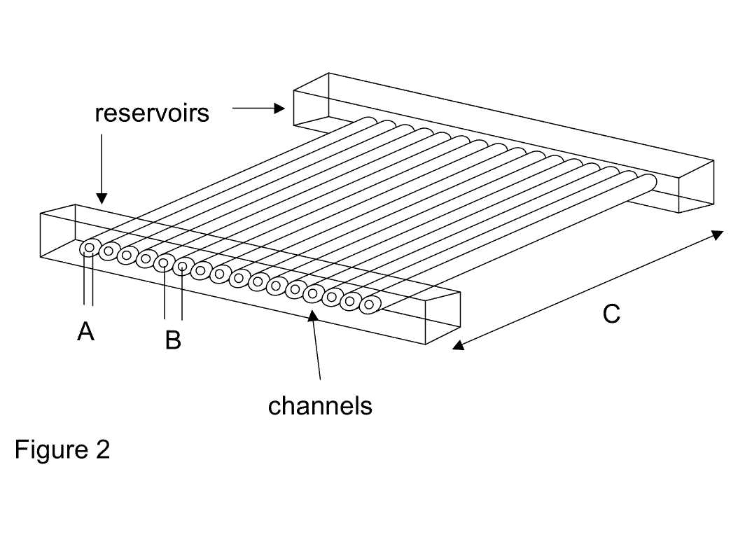

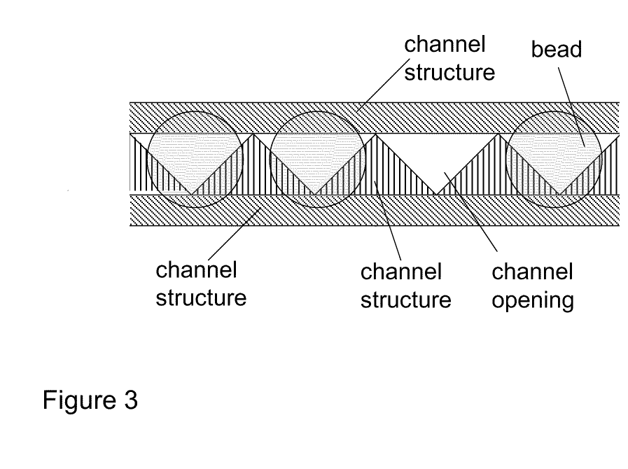

The present teachings provide systems for nucleic acid sequencing in a highly parallel manner using particle-based, clonal amplification, particle-based injection and electrophoretic size-based separation. A set of nucleic acids in a sample is clonally amplified such that individual templates are associated with single particles. Clonal amplification can occur for example by emulsion PCR. The association of the nucleic acids with the particles can be through physical adsorption, covalent bonds or non-covalent binding. Optionally, the particles can be enriched for those particles containing amplified nucleic acid. Typically, as seen in FIG. 1, a series of labeled fragments which differ in size by one nucleotide is formed by a Sanger sequencing reaction or asymmetric PCR.¹⁰ The set of particles is added to a device (see the example shown in FIG. 2) containing a multitude of channels containing a medium for size based electrophoretic separation such as a sieving polymer matrix or a cross-linked gel. Typically, a device with 1,000 to 10,000 channels is used. This type of device produces one to two orders of magnitude higher throughout in separation compared to the current state-of-the-art of capillary electrophoresis and microfluidics.¹¹ The particles are then aligned with the entrance of the channels such that most channels are associated with a single particle (see the example shown in FIG. 3). The particle-bound nucleic acids are then dissociated from the particle. For example, such release can be by photolysis of a particle-nucleic acid linkage¹², by chemical or enzymatic cleavage¹³ of a particle-nucleic acid linkage or by melting non-covalently bound nucleic acids from the particle. During the release or soon after the release, an electric field is applied across the channels. The released nucleic acids are then subjected to electrophoretic, size-based separation, and the nucleic acid fragments in each channel are optically detected after separation. The resulting electropherograms are analyzed to define the sequences of the clonally amplified nucleic acids. The practice of the present system will employ, unless otherwise indicated, conventional methods of chemistry, biochemistry, and recombinant nucleic acid techniques, within the skill of the art. Such techniques are explained fully in the literature.¹⁴

### 1. Definitions

The following terms, as used herein, are intended to be defined as indicated below.

The singular terms “a”, “an,” and “the” as used herein include plural referents unless the content clearly dictates otherwise. Thus, for example, reference to “a nucleic acid” includes a mixture of two or more such nucleic acids, and the like.

The terms “polynucleotide,” “oligonucleotide,” “nucleic acid,” and “nucleic acid molecule” are used herein to include a polymeric form of nucleotides of any length, either ribonucleotides or deoxyribonucleotides. This term refers only to the primary structure of these molecules. Thus, the term includes triple-, double-, and single-stranded RNA and triple-, double-, and single-stranded DNA. It also includes modifications of these molecules, such as by methylation and/or by capping, and unmodified forms of the polynucleotide. There is no intended distinction in length between the terms “polynucleotide,” “oligonucleotide,” “nucleic acid,” and “nucleic acid molecule,” and these terms will be used interchangeably. Thus, these terms include, for example, double- and single-stranded DNA, as well as double- and single-stranded RNA, DNA:RNA hybrids, and also include known types of modifications, for example, labels which are known in the art, methylation, “caps,” substitution of one or more of the naturally occurring nucleotides with an analog, as well as unmodified forms of the polynucleotide or oligonucleotide.

The terms “hybridize” and “hybridization” as used herein refer to the formation of complexes between nucleotide sequences that are sufficiently complementary to form complexes via Watson-Crick base pairing.

The term “particle” as used herein includes beads (for example those made from glass, quartz or polymers) liposomes, highly branched polymers and oil droplets. The term “aligning” as used herein refers to positioning a particle with respect to a channel such that a particle is near an entrance to a channel so that most of the nucleic acids released from a particle that enter a channel enter the channel with which the particle is aligned.

The term “channel” as used herein refers to a structure which connects two reservoirs and through which materials may be electrokinetically transported. Typically, such channels will include at least one cross sectional dimension that is in the range of from about 0.1 um to about 500 um, and preferably from about 1 um to about 100 um. Dimensions may also range from about 5 um to about 50 um. Typically channels are 2 cm to 80 cm long. For example, channels can be those used in microfluidic devices or capillaries used for capillary electrophoresis.

The terms “label” and “detectable label” as used herein refer to a molecule capable of being optically detected, including, absorbers, fluorescers, phosphoresers, and chemiluminescers. Such labels can be associated with nucleic acids through covalent bonding or non-covalent binding. The term “fluorescer” refers to a substance or a portion thereof which is capable of exhibiting fluorescence in the detectable range. Particular examples of labels which may be used include, but are not limited to, ethidium bromide, SYBR green, SYBR gold, fluorescein, SYTO-9, SYTO-13, SYTO-16, SYTO-60, SYTO-62, SYTO-64, SYTO-82, PO-PRO-3, YO-PRO-1, SYTOX Orange, and TO-PRO-3, FITC, rhodamine, dansyl, umbelliferone, dimethyl acridinium ester (DMAE), Texas red, luminol, NADPH, Cy3, Cy3.5, Cy5, Cy5.5, Cy7, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700 and Alexa Fluor 750, bodipy dyes, rhodamine derivatives and fluorescein derivatives etc.¹⁵ Energy transfer fluorophores can also be used.¹⁶ In addition, fluorophore labeled dideoxy terminators such as those sold by ABI including the Big Dyes can be used.¹⁷

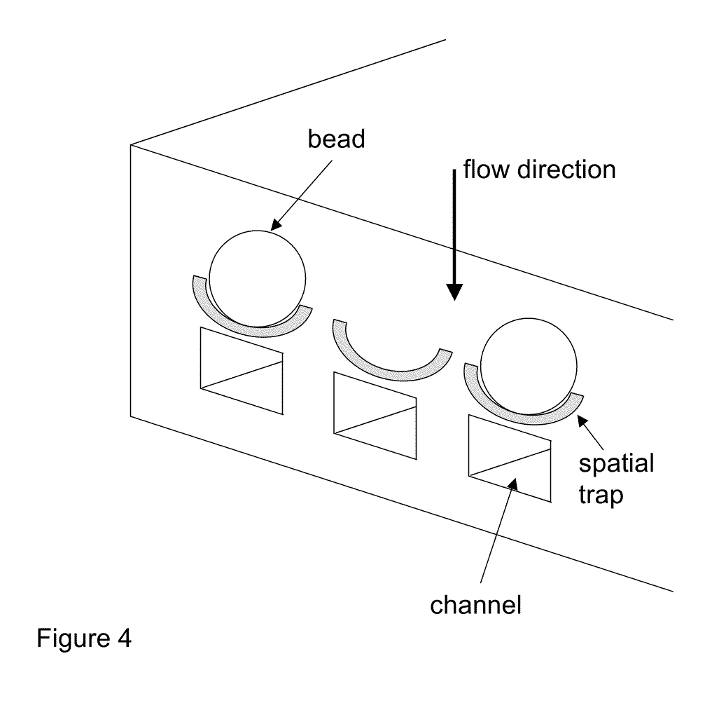

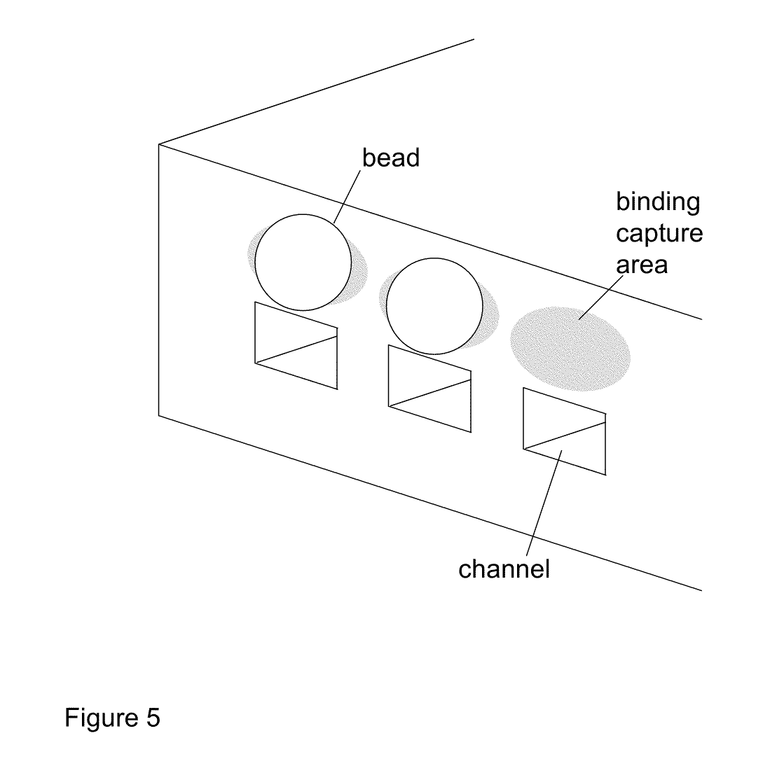

The term “spatial trap” as used herein refers to a structure which is capable of aligning a particle with a channel. For example, spatial traps could be structures as described by Lee et al.¹⁸, which are positioned near the ends of the channels (see FIG. 4). In another example, a spatial trap could be a hole in one side of the channel which allows one particle to be confined within the hole. In another example, a spatial trap could be a region near the end of a channel which is functionalized such that it can bind one particle (see FIG. 5).

The term “size-based electrophoretic separation” as used herein refers to a process in which nucleic acids which differ in size (i.e. the number of bases) travel at differing speeds in the presence of an electric field. For example, size-dependent differences in electrophoretic mobility resulting from the presence of a gel, polymer solution or similar structures or to drag-tags can be used.¹⁹ Nucleic acids which differ in size will travel at different speeds allowing for measurement of the distribution of target nucleic acid fragment sizes.

### 2. General

The present disclosure is based on novel methods for sequencing nucleic acids. The methods disclosed herein include methods for highly parallel Sanger sequencing. Parallelization is achieved by performing clonal isolation, amplification and dideoxy nucleotide termination using particle bound nucleic acids. Furthermore, the particles are aligned with a set of channels in a novel device such that most channels are associated with a single bead. Once the particles are aligned, the nucleic acids bound to the particles are released, and introduced into the channels using electrophoresis. The nucleic acids are size-separated by electrophoresis in the channels. After separation, optical signals from the nucleic acids in the set of channels are used to detect the nucleic acids. Detection can occur either within the channels or after the nucleic acid exits the channels. The set of channels is designed via materials engineering and geometric considerations to enhance signal-to-noise of the optical signals. For example, index of refraction matched or light absorbing materials can be used to reduce reflections or scattered light. The resulting set of electropherograms is analyzed to yield the sequences of nucleic acids. The methods described herein may decrease the cost of Sanger sequencing while allowing for highly parallel long read sequencing. These methods have wide ranging applicability particularly in molecular diagnostics and molecular biology research applications.

A more detailed discussion is provided below regarding the methods for highly parallel Sanger sequencing.

The method may be used with many ways of attaining clonal, in vitro amplification of nucleic acids including emulsion PCR and bridge PCR. The means by which templates are bound to the particles can vary. For example, target nucleic acids can be bound to particles by sequence specific capture oligos. In another example, nucleic acids can be sheared, size fractionated, ligated with adaptors and captured by oligos on the particles which hybridize with the adaptors or non-covalently or covalently bound to the particles.

The means for generation of dideoxy nucleotide termination fragments can vary. For example, asymmetric PCR in the presence of dideoxy nucleotide terminators can be performed in the emulsion PCR step. In another example, after emulsion PCR and breaking of the emulsion a cycle sequencing reaction can be performed that results in particle bound dideoxy nucleotide terminated fragments.

The channels can be made by a variety of methods and in a variety of sizes. For example, the set of channels can be made by bundling a set of capillaries. In another example, the set of channels can be made from planar quartz, glass or plastic substrates as is often done for microfluidic chips.²⁰ Another example involves sandwiching a micro-replicated plastic structure made of PET-backed PMMA with 25- to 50-mm pitch between channels (Vikuiti BEF II film, 3M), currently mass-produced inexpensively as a brightness enhancement structure for LCD screens, in a cartridge. The outer sandwich material on one side of the cartridge is optically transparent for fluorescent detection. To circumvent the high autofluorescence of the PET backing, a post-channel detection method can be used. Fluorescence detection can take place downstream of the channels. Either hydrodynamic sheath flow as is used in capillary arrays and/or electrophoresis can be used to transport the fluorescent bands past the detector. The design of the microfluidic device and the selection of materials are typically based on minimization of background fluorescence and optical cross-talk between channels. The cross sectional dimensions of the channels can be between 1 um and 500 um. Typically, the dimensions are between 3 and 50 um. The length of the channels can vary from 3 cm to 100 cm. Typically, the length is between 5 and 50 cm. Small center-to-center channel distances are preferred. The spacing can be between 1 and 500 um. Typically, the spacing is between 3 and 50 um. There can be 10 to 100,000 parallel channels. Typically, 2,500 to 10,000 channels are used.

Aligning the particles with the channels can be accomplished in a variety of ways. For example, the particles can be added to one of the channel reservoirs while an electric field is applied between the reservoirs. The charge on the particles causes individual particles to travel to the channel openings. The particles are prevented from entering the channels due to the relative size and shape of the particles relative to the channels. Alternatively, dielectrophoresis can be used to position the particles with respect to the channels. A hydrodynamic flow can be used to ensure that only properly aligned particles remain near the channels. In another example, spatial traps can be placed near or at the channel entrances such that particles are either physically confined to an area near the channel entrance or bind to capture molecules placed near the channel entrances (see FIGS. 4 and 5). In another example, particles can be drawn to the channel entrance by hydrodynamic flow into the channel opening.²¹ The particles are prevented from entering the channels due to the relative size and shape of the particles relative to the channels.

The nucleic acids can be released from the particles and introduced into the channels in a variety of means. For example, the nucleic acids can be bound to the particles through photolyzable, chemically cleavable or enzymatically cleavable linkages such that either illumination or addition of the appropriate chemical or enzymatic agent leads to release of the nucleic acids. In another example, nucleic acids bound to the particles through hybridization to capture oligos can be heated beyond their melting temperature to release the nucleic acids. Releasing the nucleic acids and introducing them into the channels can be done either serially or in parallel. Introduction of the nucleic acid into the channel can be done either by applying an electric field from one end of a channel to the other end or by a hydrodynamic flow into the channels. The field or flow can be applied either during the release or soon after the release. Typically, the introduction occurs less than 5 minutes and preferably less than 20 seconds after the release.

Converting each of the electropherograms to sequences can be accomplished with commercially available base calling programs such as PHRED or Sequencing Analysis Software with KB™ Basecaller available from Applied Biosystems.²²

The nucleic acids are separated in a channel. One method to perform the separation is by electrophoresis through a sieving matrix or crosslinked gel.²³ A wide variety of sieving matrices is available and can be used in this method. For example, a variety of sieving matrixes, partition matrixes and the like are available from Supelco, Inc. (Bellefonte, Pa.; see, 1997 Suppleco catalogue). Sieving matrixes typically include one or more of the following polymers: acrylamide, agarose, methyl cellulose, polyethylene oxide, hydroxycellulose, hydroxy ethyl cellulose, or the like. Combinations of any of these polymers are also optionally used. Various types of acrylamide are used, including, but not limited to, linear acrylamide, polyacrylamide, polydimethylacrylamide, polydimethylacrylamide/coacrylic acid, or the like.

Gel electrophoresis media include agarose based gels, various forms of acrylamide based gels (reagents available from, e.g., ABI, Polysciences, Suppleco, SIGMA, Aldrich, SIGMA-Aldrich and many other sources), colloidial solutions such as protein colloids (gelatins), and hydrated starches.

Many available methods for detecting nucleic acids can be used in the methods of the present disclosure. Common approaches include detection of intercalating dyes (e.g., ethidium bromide or SYBR green), detection of labels incorporated into primers used for amplification, and/or detection of labeled dideoxy nucleotides. Details of these general approaches are found in the references cited herein.²⁴

Fluorescence detection is especially preferred and generally used for detection. The detector can monitor a single type of signal, or, e.g., simultaneously monitor multiple different signals. Exemplary detectors include photomultiplier tubes, spectrophotometers, CCD arrays, microbolometers, scanning detectors, microscopes, galvo-scanns, and/or the like. Probes or other components that emit a detectable signal can be flowed past the detector. Alternatively, or in addition, the detector can move relative to the site of the probes (or, the detector can simultaneously monitor a number of spatial positions corresponding to channel regions, e.g., as in a CCD array).

The detector can include or be operably linked to a computer (or other logic device), e.g., which has software for converting detector signal information into assay result information (e.g., the length of a nucleic acid of interest), or the like.

Optical detection systems include systems that are capable of measuring the light emitted from the material within the separation device, the transmissivity or absorbance of the material, as well as the materials spectral characteristics. In preferred aspects, the detector measures an amount of light emitted from the material, such as a fluorescent or chemiluminescent material. As such, the detection system will typically include collection optics for gathering a light-based signal transmitted through the detection window, and transmitting that signal to an appropriate light detector. Microscope objectives or lenses of varying power, field diameter, and focal length can be readily utilized as at least a portion of this optical train. The light detectors are optionally spectrophotometers, photodiodes, avalanche photodiodes, photomultiplier tubes, diode arrays, or in some cases, imaging systems, such as charged coupled devices (CCDs) and the like. The detection system is typically coupled to a computer, via an analog to digital or digital to analog converter, for transmitting detected light data to the computer for analysis, storage and data manipulation.

In the case of fluorescent materials the detector typically includes a light source that produces light at an appropriate wavelength for activating the fluorescent material, as well as optics for directing the light source through the detection window to the material contained in the channel. The light source can be any number of light sources that provides an appropriate wavelength, including lasers, laser diodes, and LEDs. Other light sources are used in other detection systems. For example, broad band light sources are typically used in light scattering/transmissivity detection schemes, and the like.

### EXAMPLES

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but allowances should be made for some experimental error and deviation.

### Example 1

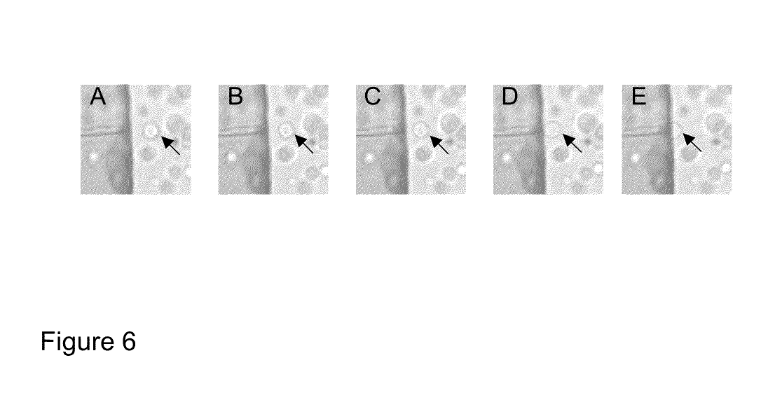

The ability to align individual beads with channels was demonstrated using a model system. Quartz capillaries (6 cm long, 363 um OD, 20 um ID, Polymicro) were filled with 100 mM TBE buffer and each end was placed in a buffer reservoir. Agarose beads (25 um mean diameter, GE Healthcare) were flowed into the upstream reservoir. The capillary ends were imaged on a Nikon Diaphot 300 microscope and video collected on an LCL 903HS CCD camera (Watec America). As shown in FIG. 6, when an electric field (167 V/cm) was applied the beads were aligned with the channels. The electrophoretic force acting on the negatively charged beads forced the beads to stick on the top of each channel (but not squeeze into the channel) since the beads were slightly larger than the opening of the channel. Once one bead is associated with one channel, steric hindrance prevents another bead from being tightly captured by the same channel. A small hydrodynamic force (flow velocity <200 um/sec) orthogonal to the channel was able to dislodge excess loosely associated beads.

### Example 2

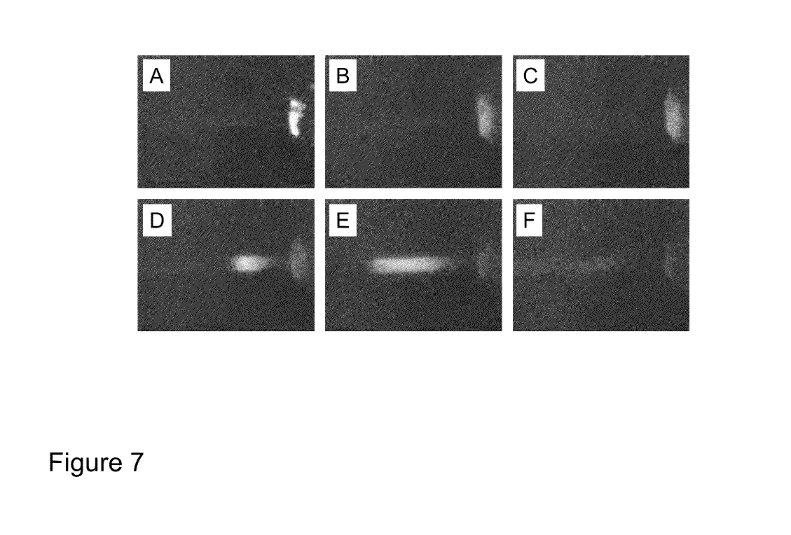

Preliminary studies to demonstrate the feasibility of releasing DNA fragments from beads and injecting them into channels were conducted. Small fluorescein and biotin labeled DNA oligos were bound to streptavidin coated glass beads (6 um diameter, Polysciences) in TBE buffer. After 30 minutes, the beads were washed to remove unbound oligos. The beads were loaded by gravity driven flow into a planar quartz microfluidic chip (AMS90 DNA chip, Caliper Life Sciences). An electric field (50 V/cm) was applied using an Agilent Bioanalyzer connected to the wells of the chip. The beads were driven from the deep (25 um) waste channel to the junction of the shallow (3 um) side channel resulting in the stacking of the beads at the entrance to the shallow channel. The fluorescein labeled oligos were imaged on a Nikon Diaphot 300 microscope (20× objective) with a mercury arc lamp using a fluorescein filter cube, and images were captured on an LCL 903HS CCD camera (Watec America). The bead/DNA linkage was photolyzed by switching to an empty filter cube with a DAPI dichroic mirror for 15 seconds. As seen in FIG. 7, the electric field caused the released oligos to be injected into the shallow channel where they were electrokinetically driven away from the injection site.

### REFERENCES

¹ Sanger F, Nicklen S, Coulson A R. DNA sequencing with chain-terminating inhibitors. Proc Natl Acad Sci USA. 1977 December; 74 (12):5463-7.
² Smith L M, Sanders J Z, Kaiser R J, Hughes P, Dodd C, Connell C R, Heiner C, Kent S B, Hood L E. Fluorescence detection in automated DNA sequence analysis. Nature. 1986 Jun. 12-18; 321 (6071):674-9. Rosenblum B B, Lee L G, Spurgeon S L, Khan S H, Menchen S M, Heiner C R, Chen S M. Nucleic Acids Res. 1997 Nov. 15; 25 (22):4500-4. Links New dye-labeled terminators for improved DNA sequencing patterns.
³ Dressman, D., Yan, H., Traverso, G., Kinzler, K. W. & Vogelstein, B. Transforming single DNA molecules into fluorescent magnetic particles for detection and enumeration of genetic variations. Proc. Natl. Acad. Sci. USA 100,8817-8822 (2003). Tawfik D S, Griffiths A D. Man-made cell-like compartments for molecular evolution. Nat Biotechnol. 1998 July; 16 (7):652-6.
⁴ M. Margulies, et al. (2005). “Genome sequencing in microfabricated high-density picolitre reactors”. Nature 437: 376-380. J. Shendure, G. J. Porreca, N. B. Reppas, X. Lin, J. Pe McCutcheon, A. M. Rosenbaum, M. D. Wang, K. Zhang, R. D. Mitra and G. M. Church (2005). “Accurate Multiplex Polony Sequencing of an Evolved Bacterial Genome”. Science 309 (5741): 1728-1732.
⁵ Adessi, C. et al. Solid phase DNA amplification: characterisation of primer attachment and amplification mechanisms. Nucleic Acids Res. 28, e87 (2000). Fedurco, M., Romieu, A., Williams, S., Lawrence, I. & Turcatti, G. BTA, a novel reagent for DNA attachment on glass and efficient generation of solid-phase amplified DNA colonies. Nucleic Acids Res. 34, e22 (2006)
⁶ Fedurco, M., Romieu, A., Williams, S., Lawrence, I. & Turcatti, G. BTA, a novel reagent for DNA attachment on glass and efficient generation of solid-phase amplified DNA colonies. Nucleic Acids Res. 34, e22 (2006). Turcatti, G., Romieu, A., Fedurco, M. & Tairi, A. P. A new class of cleavable fluorescent nucleotides: synthesis and optimization as reversible terminators for DNA sequencing by synthesis. Nucleic Acids Res. 36, e25 (2008).
⁷ Shendure, J. et al. Accurate multiplex polony sequencing of an evolved bacterial genome. Science 309, 1728-1732 (2005). McKernan, K., Blanchard, A., Kotler, L. & Costa, G. Reagents, methods, and libraries for bead-based sequencing. US patent application 20080003571 (2006).
⁸ Quinn N L, Levenkova N, Chow W, Bouffard P, Boroevich K A, Knight J R, Jarvie T P, Lubieniecki K P, Desany B A, Koop B F, Harkins T T, Davidson W S. BMC Genomics. 2008 Aug. 28; 9:404. Assessing the feasibility of GS FLX Pyrosequencing for sequencing the Atlantic salmon genome.
⁹ Palmer S, Kearney M, Maldarelli F, Halvas E K, Bixby C J, Bazmi H, Rock D, Falloon J, Davey R T Jr, Dewar R L, Metcalf J A, Hammer S, Mellors J W, Coffin J M. Multiple, linked human immunodeficiency virus type 1 drug resistance mutations in treatment-experienced patients are missed by standard genotype analysis. J Clin Microbiol. 2005 January; 43 (1):406-13. Topaloglu O, Civin C I, Bunz F. Digital HLA allelotyping. Cancer Biol Ther. 2004 September; 3 (9):899-902.
¹⁰ Sanger F, Nicklen S, Coulson A R. DNA sequencing with chain-terminating inhibitors. Proc Natl Acad Sci USA. 1977 December; 74 (12):5463-7. Smith L M, Sanders J Z, Kaiser R J, Hughes P, Dodd C, Connell C R, Heiner C, Kent S B, Hood L E. Fluorescence detection in automated DNA sequence analysis. Nature. 1986 Jun. 12-18; 321 (6071):674-9. Rao V B, Saunders N B. A rapid polymerase-chain-reaction-directed sequencing strategy using a thermostable DNA polymerase from Thermus flavus. Gene. 1992 Apr. 1; 113 (1):17-23.
¹¹ Blazej R G, Kumaresan P, Mathies R A. Microfabricated bioprocessor for integrated nanoliter-scale Sanger DNA sequencing. Proc Natl Acad Sci USA. 2006 May 9; 103 (19):7240-5.
¹² Bai X, Kim S, Li Z, Turro N J, Ju J. Design and synthesis of a photocleavable biotinylated nucleotide for DNA analysis by mass spectrometry. Nucleic Acids Res. 2004 Jan. 26; 32 (2):535-41.
¹³ Bioconjugate Techniques, 2nd Edition By Greg T. Hermanson, Published by Academic Press, Inc., 2008.
¹⁴ See, e.g., A. L. Lehninger, Biochemistry (Worth Publishers, Inc., current addition); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); and Methods In Enzymology (S. Colowick and N. Kaplan eds., Academic Press, Inc.); Sambrook et al., Molecular Cloning—A Laboratory Manual (3rd Ed.), Vol. 1-3, Cold Spring Harbor Laboratory, Cold Spring Harbor, N.Y., 2000 (“Sambrook”).
¹⁵ Haugland (2001) Handbook of Fluorescent Probes and Research Chemicals Eighth Edition by Molecular Probes, Inc. (Eugene Oreg.).
¹⁶ Lee et al. U.S. Pat. No. 5,800,996. Lee et al. U.S. Pat. No. 5,863,727. Lee et al. U.S. Pat. No. 5,945,526.
¹⁷ Lee L G, Spurgeon S L, Heiner C R, Benson S C, Rosenblum B B, Menchen S M, Graham R J, Constantinescu A, Upadhya K G, Cassel J M. New energy transfer dyes for DNA sequencing. Nucleic Acids Res. 1997 Jul. 15; 25 (14):2816-22.
¹⁸ Di Carlo D, Aghdam N, Lee L P. Single-cell enzyme concentrations, kinetics, and inhibition analysis using high-density hydrodynamic cell isolation arrays. Anal Chem. 2006 Jul. 15; 78 (14):4925-30.
¹⁹ Meagher R J, Won J I, McCormick L C, Nedelcu S, Bertrand M M, Bertram J L, Drouin G, Barron A E, Slater G W. End-labeled free-solution electrophoresis of DNA. Electrophoresis. 2005 January; 26 (2):331-50.
²⁰ Bousse L, Cohen C, Nikiforov T, Chow A, Kopf-Sill A R, Dubrow R, Parce J W. Electrokinetically controlled microfluidic analysis systems. Annu Rev Biophys Biomol Struct. 2000; 29:155-81.
²¹ Schroeder K, Neagle B, Trezise D J, Worley J. Ionworks H T: a new high-throughput electrophysiology measurement platform. J Biomol Screen. 2003 February; 8 (1):50-64.
²² Ewing B, Hillier L, Wendl M C, Green P. Base-calling of automated sequencer traces using phred. I. Accuracy assessment. Genome Res. 1998 March; 8 (3):175-85. Ewing B, Green P. Base-calling of automated sequencer traces using phred. II. Error probabilities. Genome Res. 1998 March; 8 (3):186-94.
²³ For a discussion of electrophoresis see, e.g., Weiss (1995) Ion Chromatography VCH Publishers Inc.; Baker (1995) Capillary Electrophoresis John Wiley and Sons; Kuhn (1993) Capillary Electrophoresis: Principles and Practice Springer Verlag; Righetti (1996) Capillary Electrophoresis in Analytical Biotechnology CRC Press; Hill (1992) Detectors for Capillary Chromatography John Wiley and Sons.
²⁴ See, e.g., Sambrook (2000), Ausubel (2002). Additional labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.).

What is claimed is:

1) A method for sequencing nucleic acids, the method comprising:
i) Generating a set of particles which are associated with clonally amplified nucleic acids;
ii) Aligning the particles with a set of channels such that most channels are associated with a single particle;
iii) Releasing the nucleic acids from the particles and introducing them into the channels;
iv) Separating the released nucleic acids using size-based electrophoretic separation;
v) Detecting a signal from a detectable label on the nucleic acids after separation; and
vi) Analyzing the resulting electropherograms to define the nucleic acid sequences.

2) The method of claim 1 wherein the bound amplicons are dideoxynucleotide terminated fragments generated using fluorescently labeled dideoxynucleotides.

3) The method of claim 1 wherein the set of particles is generated by emulsion PCR.

4) The method of claim 1 wherein asymmetric PCR in the presence of dideoxy nucleotides is performed.

5) The method of claim 3 wherein a cycle sequencing reaction is performed after the emulsion PCR.

6) The method of claim 1 wherein the particles are aligned by suspending the particles near the ends of the channels and applying an electric field across the length of the channels.

7) The method of claim 1 wherein the particles are aligned by suspending the particles near the ends of the channels and causing a hydrodynamic flow into the channel ends.

8) The method of claim 1 wherein the particles are aligned by spatially trapping the particles.

9) The method of claim 1 wherein the nucleic acids are released by photolysis of a linkage between the particle and the nucleic acids.

10) The method of claim 1 wherein the nucleic acids are released by heating the particles to cause the nucleic acids to melt from oligos attached to the particles.

11) The method of claim 1 wherein the nucleic acids are released by chemical or enzymatic cleavage of a linkage between the nucleic acids and the particles.

12) The method of claim 1 wherein the particles are beads, liposomes or oil droplets.

13) The method of claim 1 wherein there are more than 1,000, 10,000 or 100,000 channels.

14) The method of claim 1 wherein the channels are straight channels made in a polymeric device.

15) The method of claim 1 wherein size-based electrophoretic separation is performed using cross-linked gels.

16) The method of claim 1 wherein size-based electrophoretic separation is performed using a polymer matrix.

17) The method of claim 1 wherein detection occurs after the nucleic acids leave the separation channels.

18) The method of claim 17 wherein the nucleic acid is transported out of the channel solely by electrophoresis.

19) A method for sequencing nucleic acids, the method comprising:
i) Generating a set of beads which are associated with clonally amplified, fluorescent dideoxy nucleotide terminated nucleic acids;
ii) Aligning the beads using electrophoresis with a set of channels such that most channels are associated with a single bead;
iii) Releasing the nucleic acids from the beads by photolysis and introducing the nucleic acids into the channels;
iv) Separating the released nucleic acids using size-based electrophoretic separation;
v) Detecting fluorescence from the dideoxy terminated fragments after separation; and
vi) Analyzing the resulting electropherograms to define the nucleic acid sequences.

20) A method for sequencing nucleic acids, the method comprising:
i) Generating a set of beads which are associated with clonally amplified, fluorescent dideoxy nucleotide terminated nucleic acids by emulsion PCR followed by a cycle sequencing reaction;
ii) Aligning the beads using electrophoresis with a set of channels such that most channels are associated with a single bead;
iii) Releasing the nucleic acids from the beads by photolysis and introducing the nucleic acids into the channels by electrophoresis;
iv) Separating the released nucleic acids using size-based electrophoretic separation;
v) Detecting fluorescence from the dideoxy terminated fragments after separation; and
vi) Analyzing the resulting electropherograms to define the nucleic acid sequences.
